(19) United States
(12) Patent Application Publication
(10) Pub. No.: US 2011/0268808 A1
(43) Pub. Date: Nov. 3, 2011

(54) DUAL-RELEASE PHARMACEUTICAL SUSPENSION

(75) Inventors: Rajesh Jain, New Delhi (IN); Sukhjeet Singh, New Delhi (IN); Sanju Dhawan, New Delhi (IN)

(73) Assignee: PANACEA BIOTEC LTD., New Delhi (IN)

(21) Appl. No.: 13/143,884

(22) PCT Filed: Jan. 8, 2010
(86) PCT No.: PCT/IN10/00013

§ 371 Date: Jul. 8, 2011

(30) Foreign Application Priority Data
Jan. 9, 2009 (IN) 30/DEL/2009

(51) Int. Cl. A61K 9/16; A61K 31/192; A61K 31/397; A61K 31/497; A61P 9/00; A61P 31/04; A61P 9/12; A61P 9/04; A61P 25/00; A61P 25/08; A61K 31/167; A61P 31/12

(52) U.S. Cl. 424/497; 514/630; 514/569; 514/210.05; 514/253.08

(57) ABSTRACT

Orally deliverable dual-release pharmaceutical suspensions, having a first portion comprising an immediate release form of the active in the solution form or granules or suspended form in the vehicle/medium preferably in the solution form and a second portion comprising a sustained-release form of active in the form of microgranules/microparticles suspended in the immediate release fraction of the solulabilised active agent which comprise a core and at least one coat suitable for liquid dosage forms for the administration of the active ingredients, wherein the core comprises at least one active agent(s) or its pharmaceutically acceptable salts, derivatives, isomers, polymorphs, solvates, hydrates, analogues, enantiomers, tautomeric forms or mixtures thereof; optionally at least one water insoluble, and optionally one or more pharmaceutically acceptable excipient(s); and at least one coat comprising at least one pH independent water-insoluble polymer(s) along with one or more pharmaceutically acceptable excipient(s). This coated microparticles and solution of the active agent in the vehicle ensures a dual release profile i.e. immediate release profile as well as predetermined sustained release profile of the active agent and also ensures maintenance of said release profile over time. The present invention can be administered either in the form of ready to use suspension or in the form of powder ready for reconstitution. Further, this invention provides process of preparation of such suspensions and method of using them.

### FIELD OF THE INVENTION

The present invention describes orally deliverable dual-release pharmaceutical suspensions, having a first portion comprising an immediate release form of the active in the solution form or microgranules or suspended form in the vehicle/medium and a second portion comprising a sustained-release form of active in the form of microgranules/microparticles which comprise a core and at least one coat wherein the core comprises at least one active agent(s) or its pharmaceutically acceptable salts, derivatives, isomers, polymorphs, solvates, hydrates, analogues, enantiomers, tautomeric forms or mixtures thereof; optionally at least one water-insoluble polymer(s), and optionally one or more pharmaceutically acceptable excipient(s); and at least one coat comprising at least one pH independent water-insoluble polymer(s) along with one or more pharmaceutically acceptable excipient(s). This coated microparticles and solution of the active agent in the vehicle ensures a dual release profile i.e. immediate release profile as well as predetermined sustained release profile of the active agent and also ensures maintenance of said release profile over time. The present invention can be administered either in the form of ready to use suspension or in the form of powder ready for reconstitution. Further, this invention provides process of preparation of such suspensions and method of using them.

### BACKGROUND OF THE INVENTION

A controlled release preparation is one that achieves slow release of a drug over an extended period of time, thereby extending the duration of drug action over that achieved by conventional delivery. Drugs that are administered multiple times per day and drugs with high inter- and/or intra-patient variability can be more therapeutic efficient if administered as controlled release formulation. The advantages of controlled release products are well-known in the pharmaceutical field and include the ability to release the medicament in a controlled manner over a period of time while increasing patient compliance by reducing the number of administrations necessary to achieve the same level. Several controlled release compositions for delivering different pharmaceutically active ingredients and involving different release mechanisms had been described previously. In addition, multiple dose liquid formulations are preferable to tablets or capsules because formulations like tablets or capsules cannot be administered to patients with swallowing difficulties and to children or infants, who in any case are incapable of swallowing and for whom in addition, the dose administered has to be adapted according to their weight.

U.S. Pat. No. 6,958,161 describes a modified release preparation having one or more coated core elements, each core element comprising an active ingredient selected from the group consisting of the acid salts of doxycycline, tetracycline, oxytetracycline, minocycline, chlortetracycline or demeclocycline and having a modified release coating, wherein a stabilizing coat is provided between each core element and its modified release coating so that, upon in vitro dissolution testing, the amount of active ingredient released at any time on a post-storage dissolution profile is within 40 percentage points of the amount of active ingredient released at any time on a pre-storage dissolution profile. U.S. Pat. No. 6,932,981 describes a fast disintegrating controlled release oral composition comprising a core material containing cefuroxime axetil present as controlled release form, and optionally probenecid, said controlled release form comprising a) an outer coating of a polymer selected from aqueous dispersions of enteric methacrylic acid and methacrylic acid esters anionic copolymers having carboxygroup as the functional group or mixtures thereof and; b) an inner coating of a sustained-release copolymer selected from aqueous dispersions of acrylate and methacrylate pH independent, neutral copolymers having quaternary ammonium group as a functional group or mixtures thereof; said composition releases cefuroxime axetil in amounts of more than 80% in 4 hours and the outer coating controls the initial rapid release of cefuroxime axetil from the composition. U.S. Pat. No. 5,968,554 describes an oral dosage delivery form adapted to deliver a pH dependent water soluble therapeutic agent comprising: (a) a core comprising said therapeutic agent in an amount sufficient to deliver from 25-75% of an effective amount of said therapeutic agent over the intended delivery time; (b) an enteric polymer coating over said core; (c) a coating of said therapeutic agent over said enteric polymer coating in an amount sufficient to deliver from 25-75% of an effective amount of said therapeutic agent over the intended delivery time; and (d) a low pH soluble protective coating over said coating of said therapeutic agent. PCT publication no. WO200693838 claims a method for preparing a liquid, controlled-release formulation comprising the steps of blending one or more controlled release microbeads comprising one or more active agents; preparing a dense, thixotropic solution having a density that is at or about the density of the one or more microbeads comprising a thixotropic agent, water and one or more preservatives under conditions that reduce bubble formation; and mixing the microbeads and the thixotropic solutions under conditions that minimize the introduction of bubbles in the liquid. U.S. Pat. No. 4,717,713 discloses in-situ gelling system for controlled release of drug in suspension form. Extended release of water soluble or slightly water soluble drugs for longer duration of time period (more than 10 hours) is not possible with in-situ gelling suspension formulations. PCT publication no. WO0145706 discloses a pharmaceutical composition comprising one or more orally deliverable dose units, each comprising a first fraction of a selective cyclooxygenase-2 inhibitory drug of low water solubility in an amount of about 10 mg to about 400 mg, said first fraction being in solution in a pharmaceutically acceptable solvent and/or present in immediate-release solid particles having a D50 particle size less than about 5 um; and a second fraction of the drug in an amount of about 10 mg to about 400 mg, said second fraction being present in solid particles having a particle size greater than about 25 um and/or in controlled-release, slow-release, programmed-release, timed-release, pulse-release, sustained-release or extended-release particles; wherein said first fraction and said second fraction of the drug are present in a weight ratio of about 10:1 to about 1:10. PCT publication no. WO2007138466 discloses a method of treating moderate to severe pain in a patient in need thereof, the method comprising administering a pharmaceutical composition comprising tramadol or a pharmaceutically acceptable salt or a derivative thereof and meloxicam or a pharmaceutically acceptable salt or a derivative thereof in admixture with one or more pharmaceutically acceptable carriers, wherein the composition provides extended-release of the tramadol and immediate release of the meloxicam. PCT publication no. WO2008064192 discloses a liquid pharmaceutical dosage form comprising: a) a first portion containing a first active ingredient comprised of an NSAID and/or pharmaceutically acceptable salts thereof, wherein the first active ingredient is released from the dosage form in a substantially immediate manner upon contact of the dosage form with a dissolution medium; and b) a second portion containing i. ion exchange resin particles having a second active ingredient bound thereon to form drug-resin complex particles; ii. a semi-permeable coating layer substantially covering said drug-resin complex particles to form coated particles; and iii. a protective coating layer substantially covering said coated particles, wherein the second active ingredient is released from the second portion in a modified release manner upon contact of the dosage form with the dissolution medium, and wherein the duration of the therapeutic effect of the second active ingredient as released from the second portion of the dosage form is substantially the same as the duration of the therapeutic effect of the first active ingredient.

U.S. Pat. Nos. 4,762,709 and 6,328,979, US Publication no. 2005181050 and PCT publication nos. WO 9827961 and WO 2005117843 disclose controlled release suspension technology based on ion exchange resins. Although ion exchange resins systems (such as Pennkinetic system) provide long extended drug release without significant drug leaching during storage, these systems require chemical binding of drug to the resin which is not suitable for many drugs with ease.

Oral pharmaceutical formulations in the form of tablets or capsules for the controlled release of many drugs currently exist. These formulations cannot be administered to patients with swallowing difficulties and to children or infants, who in any case are incapable of swallowing and for whom in addition, the dose administered has to be adapted according to their weight. Multiple dose liquid formulations are preferable to tablets or capsules for such applications. Drugs that are administered multiple times per day and drugs with high inter- and/or intra-patient variability can be more therapeutic efficient if administered as controlled release formulation. Liquid formulations for the controlled release of drugs are difficult to produce. The main difficulties are, avoiding of drug release into the liquid phase during storage of formulation, need of small particle size to avoid grittiness in mouth and balding of unpleasant taste of drug. Avoiding of drug release into the liquid phase during storage of formulation, while allowing controlled release of drug as soon as it enters the gastrointestinal tract is particularly difficult to achieve for water soluble or even slightly water soluble drugs because drug is stored in liquid for a very long time (about 10-15 days) compared with the desired release time in the gastrointestinal tract fluids (4-16 hours).

A common problem associated with liquid pharmaceutical dosage forms is the often disagreeable taste of a drug that may manifest itself when the drug is in a liquid dosage form. Sometimes, the taste of the drug in the dosage form may be overpowered by adding sweeteners or flavoring agents to the liquid dosage. These agents mask the bitter or unpleasant taste of drugs. However, these agents are not totally effective in concealing the unpalatable taste of pharmaceuticals.

Liquid suspension dosage forms also have stability problems associated with maintaining the drugs in suspension. Poorly formulated liquid pharmaceutical suspensions allow the drug to settle out as a sediment, thereby reducing the therapeutic concentration of drug in the suspension. This results in under dosing or over dosing of the patient, which may seriously compromise the patient's recovery.

Additionally the pharmaceutical suspension should be readily pourable so that the dosage is easy to administer. The requirement that a pharmaceutical suspension is readily pourable effectively places an upper limit on the viscosity of the suspension. This limitation also indirectly limits the amount of pharmaceutical actives that the suspension will suspend.

In view of these difficulties it would be desirable to develop a ready-to-use stable pharmaceutical suspension with a high degree of stability and good taste masking characteristics. Therefore, there exists a need for a stable suspension system for pharmaceutical actives that minimizes sedimentation of the active ingredients and provides a pleasant tasting liquid dosage.

The present invention discloses a stable aqueous suspension system for pharmaceutical actives, which can be combined with sweeteners and flavoring agents to provide a palatable liquid dosage form. This dosage form is also physicochemically stable and especially well suited for both geriatric and pediatric applications.

However none of the prior art documents described hereinabove disclose pharmaceutical compositions which are highly safe and effective and are easy to formulate as described in context of the present invention. A review of the prior art reveals that there still exists an unmet medical need for development of pharmaceutical compositions to provide treatment regimens that could alleviate the drawbacks associated with the prior art compositions that have so far plagued effective patient management and a need to develop particularly pharmaceutical compositions which can remain stable for a prolonged period of time at least for the entire duration of therapy and also provide a desired controlled release of the active agent(s).

It was surprisingly found by our formulation scientist that if sustained release granules of a drug are kept in a solution of the drug, the drug will not diffuse from the sustained release granules to the outer solution and the release profile of such a formulation will remain same for sufficient period of time to provide it good stability. The technology can be adopted for drugs where simultaneous release of immediate release and sustained release is desirable.

The dual-release pharmaceutical suspensions upon reconstitution remains stable till its consumption and also provide a dual-release of the active agent(s). The major problem with the sustained/extended release suspensions is the stable release profile of the suspension over a period of time. The release profile changes on subsequent days after reconstitution as some of the drug comes out of drug resin complex/granules into the media of the suspension as the Tussionex® Suspension which is a controlled release hydrocodone resin complex and phenyltoloxamine resin complex. Over a period of time, the formulation will behave as immediate release suspension. In order to prevent release of the drug from microgranules into the vehicle of the suspension, researchers have tried to apply pH dependent coatings. However, this increases the number of coatings on the granules and total solid content in the suspension and also leads to cumbersome process. The current investigation aims at applying pH independent coating onto the granules for obtaining sustained release fraction, the immediate release fraction is achieved by drug solution. These two drug fractions in one formulation lead to a stable release profile over a period of time.

The present invention provides suspensions to overcome the limitations of the prior art and provides safe and effective compositions for the management of disease(s) which are particularly devoid of the associated stability issues and therefore provides a significant advancement in the said field. The composition of the present invention is a liquid preparation which can be prepared by reconstitution or ready to use suspensions which gives the flexibility to the patient. The composition gives stable dual release profile over a stipulated period of time. It also masks the bitter taste of drugs, thus proving to be more patient compliant.

### SUMMARY OF THE INVENTION

It is an objective of the present invention to provide an oral dual-release pharmaceutical suspensions comprising immediate release fraction and sustained release fraction of the active agent or its pharmaceutically acceptable salts, derivatives, isomers, polymorphs, solvates, hydrates, analogues, enantiomers, tautomeric forms or mixtures thereof; wherein the said two fractions of the active agent i.e., immediate release fraction to extended release fraction is in a fixed ratio of about 20:1 to about 1:20, in admixture with one or more pharmaceutically acceptable excipient(s).

It is another objective of the present invention to provide dual-release pharmaceutical suspensions comprising immediate release fraction of active agent in the form of solution, suspension and uncoated microgranules and optionally one or more pharmaceutically acceptable excipient(s).

It is an objective of the present invention to provide dual-release pharmaceutical suspensions comprising sustained release fraction of the active agent in the form of coated microparticles which comprise a core and at least one coat, wherein the core comprises at least one active agent(s); water-insoluble polymer(s), optionally along with one or more pharmaceutically acceptable excipient(s); and at least one coat comprising at least one pH independent water-insoluble polymer(s) one or more pharmaceutically acceptable excipient(s).

It is another objective of the present invention to provide dual-release pharmaceutical suspensions wherein the said microgranules are suspended in aqueous suspending medium which contains active drug in solution form as well as in the form of immediate release uncoated microgranules.

It is another objective of the present invention to provide dual-release pharmaceutical suspension wherein the sustained release microgranules of the drug are suspended in the aqueous suspending medium in such a way that the diffusion of drug from the sustained release microgranules to the aqueous suspending medium is negligible or nearly about zero so that the dissolution profile does not change over the extended period of time.

It is another objective of the present invention to provide dual-release pharmaceutical suspension wherein the sustained release granules of the drug are suspended in the aqueous suspending medium such that the drug diffusion is dependent on drug concentration gradient formed between the coated sustained release granules and aqueous suspending medium comprising drug as well as the uncoated granules. This may lead to a stable dissolution profile of the formulation upon storage thereby increasing its shelf-life.

It is an another objective of the present invention to provide dual-release pharmaceutical suspensions comprising at least one solubility modifying agent(s) in the aqueous medium, wherein the amount of active agent (s) which is in the immediate release form in the aqueous medium can be varied between the range from about 1% to about 50% of solubilizer or solubility modifying agent.

It is objective of the present invention to provide dual-release pharmaceutical suspensions which is either in the form of ready to use suspension or in the form of powder ready for reconstitution, wherein the size of the core microparticles may range from about 1 μm to about 1000 μm.

It is another objective of the present invention to provide process for preparation of such pharmaceutical suspensions which comprises of the following steps:
i) preparation of microparticles comprising a core and at least one coat,
ii) Suspending the composition of step (i) in a suitable medium containing immediate release fraction in solution form, suspended form or in form of uncoated microparticles to obtain a dual-release suspension.

It is yet another objective of the present invention to provide a method of using such dual-release pharmaceutical suspensions which comprises administering to a subject in need thereof an effective amount of the suspensions.

### DETAILED DESCRIPTION OF THE DRAWINGS

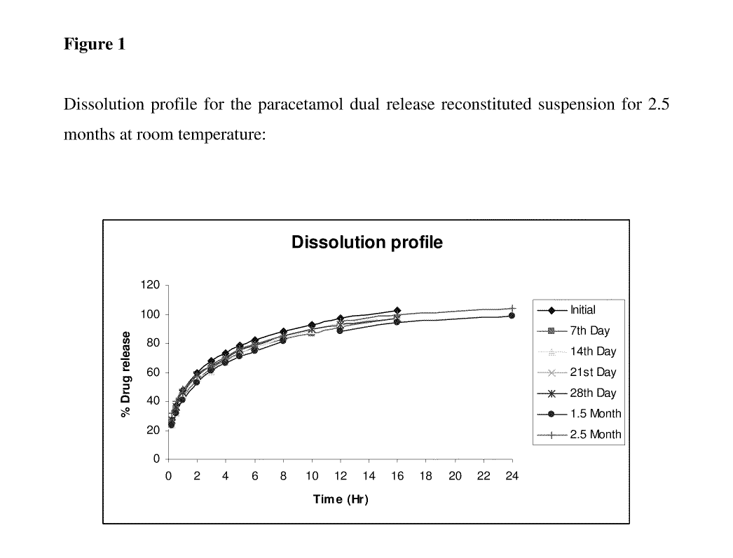

FIG. 1: The said FIGURE shows dissolution profile for the paracetamol reconstituted suspension (for the time period of 2.5 months stability data at room temperature) of the composition as mentioned hereinafter in Example-1.

### DETAILED DESCRIPTION OF THE INVENTION

The present invention provides an oral dual-release pharmaceutical suspensions comprising immediate release fraction and sustained release fraction of the active agent or its pharmaceutically acceptable salts, derivatives, isomers, polymorphs, solvates, hydrates, analogues, enantiomers, tautomeric forms or mixtures thereof; wherein the sustained release fraction and the immediate release fraction of the active agent is in a fixed ratio of about 20:1 to about 1:20, in admixture with one or more pharmaceutically acceptable excipient(s).

Active in context of present invention encompasses its pharmaceutically acceptable salts, derivatives, isomers, polymorphs, solvates, hydrates, analogues, enantiomers, tautomeric forms or mixtures thereof.

In context of the present invention “Dual-release” can be defined as a release which involves initial prompt release of the active agent for achieving early onset of action which is then followed by its sustained release for longer duration of action.

In context of the present invention, the term microparticles can be used interchangeably with the terms of microgranules, microcapsules and microspheres.

In an embodiment, the present invention provides dual-release pharmaceutical suspensions comprising immediate release fraction of active agent in the form of solution, suspension and uncoated microgranules and optionally one or more pharmaceutically acceptable excipient(s).

In another embodiment, the present invention provides dual-release pharmaceutical suspensions comprising sustained release fraction of the active agent in the form of coated microparticles which comprise a core and at least one coat, wherein the core comprises at least one active agent(s); optionally at least one water-insoluble polymer(s), optionally along with one or more pharmaceutically acceptable excipient(s); and at least one coat comprising at least one pH independent water-insoluble polymer(s) one or more pharmaceutically acceptable excipient(s), wherein the coated microgranules are suspended in the aqueous medium which also contains the immediate release fraction of active agent in the solution form.

In yet another embodiment, the present invention provides dual-release pharmaceutical suspensions wherein the said microgranules are suspended in aqueous medium which contains active drug in solution form as well as in the form of immediate release uncoated microgranules of the active agent.

In another embodiment, the present invention provides dual-release pharmaceutical suspensions comprising coated microparticles along with at least one solubility modifying agent(s) in the aqueous medium, wherein the amount of active agent (s) which is in the immediate release form in the aqueous medium can be varied by varying the amount of solubilizer or solubility modifying agent.

In preferred embodiment, the present invention relates to liquid pharmaceutical formulations like suspensions for oral administration of active agent(s) wherein the suspension releases the active agent(s) in a dual manner first as immediate followed by sustained release of the active agent does not change significantly during the storage of the liquid formulation.

The suspensions of the present invention are useful in masking the taste of bitter drugs intended for oral administration when the suspension contains sustained release coated microgranules and immediate release uncoated microgranules. Further, the dual-release pharmaceutical suspensions of the present invention remains till its consumption and also provide an immediate as well as sustained release of the active agent(s). The dual-release suspensions do not form any substantial sedimentation of the dispersed particles or a hard cake during storage for its shelf-life; instead the particles forming any loose deposits can be easily resuspended upon shaking prior to use.

In an embodiment, the active agents useful for the present invention are preferably those which have a shorter to a medium duration of therapy such as analgesics, antibiotics, anti-inflammatory drugs, antipyretics, antihistamines and the like.

In another embodiment of the present invention, the suspensions of the present invention may comprise of active agents which are useful for longer duration of therapy.

In preferred embodiment, the active agent of the present invention is selected from but not limited to a group comprising of at least one active agent(s) selected preferably from a group comprising paracetamol, nimesulide, diclofenac sodium, indomethacin, ketoprofen, diflunisal, piroxicam, naproxen, levocetirizine, desloratadine, fexofenadine, aspirin, glipizide, glyburide, glimepiride, gliclazide, metformin, rosiglitazone, pioglitazone, vildagliptin, sitagliptin, metoclopramide, diphenhydramine, loratadine, desloratadine, meclizine, quetiapine, fexofenadine, pheniramine, cetirizine, promethazine, chlorpheniramine, cimetidine, famotidine, ranitidine, nizatidine, roxatidine, lafutidine, metronidazole, chlorpromazine, fluphenazine, prochlorperazine, rimantadine, amantadine, emtricitabine, lamivudine, zidovudine, stavudine, zalcitabine, ritonavir, saquinavir, indinavir, nevirapine, ciproflocaxin, amoxicillin, voriconazole, posaconazole, oxcarbazepine, carbamazepine, phenyloin and the like or pharmaceutically acceptable salts, hydrates, polymorphs, esters and derivatives thereof used either alone or in combination thereof.

In an embodiment of the present invention, the active agent(s) comprises from about 1% to about 70% by weight of the total weight of suspension composition.

In an embodiment of the present invention, water-insoluble polymer(s) present in the core composition of the microparticle is selected from but not limited to a group comprising pH independent or mixtures thereof as described hereinafter.

In another embodiment of the present invention, the coating composition is formulated as a release controlling system that aids in providing sustained-release of the active agent(s).

In an embodiment, the release controlling system for coating the core comprises at least one pH independent water-insoluble polymer(s), and one or more other pharmaceutical excipient(s).

In an embodiment of the present invention, the microparticles consist of a matrix and are conveniently made by a hot melt granulation technique.

In an embodiment of the present invention, the lipidic agent(s) present in the core composition of the microparticle is selected from but not limited to a group comprising beeswax, carnauba wax, cetyl palmitate, Compritol® 888 ATO (glyceryl behenate), glyceryl monostearate, precirol ATO (glyceryl palmitostearate), hydrogenated castor oil, paraffin wax, stearic acid, steryl alcohol, glyceryl trimyristate (Dynasan 114), gelucire, Sterotex® K or mixtures thereof as described hereinafter.

In an embodiment, the pH independent polymer is selected from but not limited to a group comprising alkyl celluloses such as polyacrylate polymers (e.g. Eudragit® NE 30D, Eudragit® RS, Eudragit® RL) and the like or mixtures thereof.

In an embodiment, the pH independent polymer of the present invention is water-insoluble.

In another embodiment of the present invention, the pH independent water-insoluble polymer is selected from a group comprising polyacrylate polymers e.g. Eudragit® NE 30D, Eudragit® RS, Eudragit® RL or a cellulosic polymer e.g. ethylcellulose, hydroxyl ethyl cellulose, cellulose acetate or mixtures thereof.

In yet another embodiment the pH independent polymer is Eudragit® RS 30D.

In another embodiment the percentage weight gain on coating is about from 5% to about 50% by weight of the total microparticle weight.

In an embodiment of the present invention, the solubility modifying agent present in the dual-release pharmaceutical suspensions is selected from but not limited to a group comprising sugars such as but are not limited to xylose, ribose, glucose, mannose, galactose, fructose, dextrose, sucrose, maltose, partially hydrolyzed starch solids, partially hydrolyzed corn syrup solids, sorbitol, xylitol, mannitol, sodium citrate and magnesium hydroxide, weak acids such as but are not limited to fumaric acid, citric acid, tartaric acid, malic acid, maleic acid or succinic acid; amino acids such as but are not limited to L-arginine and L-lysine.

In an embodiment of the present invention, solubility modifying agents comprises in the range from about 5% to about 50% by weight of the final composition.

In an embodiment of the present invention, the in-situ gelling agent for suspension which may also be present in the dual-release pharmaceutical suspensions is selected from but not limited to a group comprising alginates such as sodium alginate; or gums such as locust bean gum, xanthan gum, tragacanth, xylan, arabinogalactan, agar, gellan gum, guar gum, apricot gum (Prunus armeniaca, L.), carrageenan, pectin, acacia gum, dextran, and gum arabic and the like, or mixtures thereof. In a preferred embodiment, sodium alginate is used as the in-situ gelling agent.

In another embodiment of the present invention, the pharmaceutically acceptable excipient(s) of the present invention are selected from but not limited to a group comprising, fillers, viscosity modifiers, anti-caking agents, thixotropic agents, anti-oxidants, colorants, flavoring agents, sweeteners, preservatives, glidants, chelating agents, plasticizers, vehicles, wetting agents, complexing agents, buffering agents, preservatives, suspending agents, release modifiers, and the like known to the art used either alone or in combination thereof. It will be appreciated that certain excipients used in the present composition can serve more than one purpose.

In an embodiment of the present invention, the viscosity enhancing agent is selected from but not limited to group comprising cellulose derivatives, such as hydroxypropyl cellulose, hydroxyethyl cellulose, hydroxypropyl methyl cellulose, methylcellulose, carboxymethyl cellulose, sodium carboxymethyl cellulose and its derivatives, microcrystalline cellulose, carbomers, or gums such as locust bean gum, xanthan gum, tragacanth, xylan, arabinogalactan, agar, gellan gum, guar gum, apricot gum, carrageenan, pectin, acacia gum, dextran, and gum arabic and the like; or mixtures thereof.

In another embodiment of the present invention, the wetting agent useful in the present invention is a surfactant selected from but not limited to a group comprising anionic, cationic, nonionic or zwitterionic surfactant, or combinations thereof. Examples of suitable wetting agents include sodium lauryl sulphate, cetrimide, polyethylene glycols; polyoxyethylene-polyoxypropylene block copolymers known as poloxamer; polyglycerin fatty acid esters such as decaglyceryl monolaurate and decaglyceryl monomyristate; sorbitan fatty acid ester such as sorbitan monostearate (SPAN® 80); polyoxyethylene sorbitan fatty acid ester such as polyoxyethylene sorbitan monooleate (TWEEN® 80, TWEEN® 40); polyethylene glycol fatty acid ester such as polyoxyethylene monostearate; polyoxyethylene alkyl ether such as polyoxyethylene lauryl ether; polyoxyethylene castor oil and hardened castor oil, such as polyoxyethylene hardened castor oil; and the like or mixtures thereof.

In another embodiment of the present invention, the preservative useful in the present invention is selected from but not limited to a group comprising parabens such as methyl paraben, propyl paraben; sodium benzoate, cetrimide and the like or mixtures thereof. Suitable glidants are selected from but not limited to a group comprising talc; stearic acid, magnesium stearate, calcium stearate, colloidal silicon dioxide such as Aerosil® 200; sodium stearyl fumarate, hydrogenated vegetable oil and the like or mixtures thereof. Suitable sweeteners include sucrose, saccharin, compressible sugar, aspartame, saccharin, or mixtures thereof. Suitable anticaking agent useful in the present invention is amorphous silica.

In an embodiment, the composition of the present invention relates to oral liquid pharmaceutical formulations like suspension which is either in the form of ready to use suspension or in the form of powder ready for reconstitution. The granulation technique is either aqueous or non-aqueous, more preferably wet granulation. In preferred embodiment, powder or granular formulations may be manufactured using techniques which are generally conventional in the field of manufacture of pharmaceutical formulations. For example a suitable technique of manufacture comprises mixing dry powdered or granulated ingredients with suitable excipient(s) and dispersing in a suitable medium to form liquid dosage composition.

In another embodiment of the present invention, the suspension may be provided in the form of a dry powder or granular substance comprising the active agent(s) and one or more excipient(s), which may be freshly prepared into the liquid suspension form by simply adding water and mixing so as to obtain a homogeneous and formulation. The granules may be prepared according to hot melt granulation technique. More preferably granules are coated with polymer system to provide formulation of drug with controlled release profile that does not change during the storage of the liquid formulation.

In another embodiment, the suspensions of the present invention can be formulated as extended release suspensions or a combination of immediate release and extended release suspensions. In another embodiment of the present invention, the size of the core microparticles may range from about 0.5 μm to about 2000 μm or from about 1 μm to about 1000 μm or from about 50 μm to about 500 μm.

In an embodiment, the present invention provides liquid taste masked controlled release pharmaceutical suspensions for oral administration of active agents which produces unpleasant or bitter taste in mouth of a subject upon administration.

In an embodiment of the present invention, the pharmaceutical dosage form composition of the present invention is formulated as an oral dosage form either as a solid, or a liquid preparation such suspension, and the like.

In preferred embodiment, powder or granular formulations may be manufactured using hot melt granulation techniques, in which drug is dispersed/dissolved in hot molten lipid and after drying, granules are sized to the required range, which are generally conventional in the field of manufacture of pharmaceutical formulations and in the manufacture of dry formulations for reconstitution into such formulations. For example a suitable technique of manufacture comprises mixing dry powdered or granulated ingredients with suitable excipient(s) and compressing to form solid dosage forms like tablets or dispersing in a suitable medium to form liquid dosage composition. In preferred embodiment, the present invention relates to liquid pharmaceutical formulations like suspensions for oral administration of active agent(s) wherein the composition releases the active agent(s) in a dual manner such that the release profile of the active agent does not change during the storage of the liquid formulation.

In an embodiment, the appropriate amount of any pharmaceutically active agent(s) in the dosage form will depend on the particular agent and/or the intended daily dose and/or the intended use. Unless explicitly indicated herein, it is to be understood that appropriate daily doses for the various agents will be known to those of ordinary skill in the art of pharmaceutical formulation and pharmacology and/or can be found in the pertinent texts and literature.

In another embodiment of the present invention is provided process for preparation of such pharmaceutical suspensions which comprises of the following steps:
i) preparation of microparticles comprising a core and at least one coat,
ii) Suspending the composition of step (i) in a suitable medium containing immediate release fraction in solution form, suspended form or in form of uncoated microparticles to obtain a dual-release suspension.

In another embodiment of the present invention is provided a process for the preparation of such composition which comprises of the following steps:
i) preparation of microparticles comprising a core and at least one coat,
ii) preparation of a controlled release composition comprising microparticles along with at least one in-situ gelling agent(s), at least one cross-linking agent(s), and optionally one or more pharmaceutically acceptable excipient(s), and
iii) optionally dispersing the composition of step (i) in a suitable reconstituting medium to obtain a controlled release suspension.

In another embodiment of the present invention is provided a process for the preparation of such composition which comprises of the following steps:
i) preparing a core composition by mixing the active agent(s) with diluent(s) and granulating with pH independent water-insoluble polymer(s) optionally with a binder,
ii) providing a first coating on the core composition with a coating composition comprising pH independent water-insoluble polymer(s) optionally along with one or more pharmaceutically acceptable excipient(s),
iii) mixing the coated granules of step (iii) with in-situ gelling agent(s), cross-linking agent(s) optionally along with one or more pharmaceutically acceptable excipient(s), and
iv) optionally dispersing the composition of step (iv) in a suitable reconstituting medium to obtain a controlled release suspension.

In yet another embodiment of the present invention is provided method of using such suspensions, which comprises administering to a subject in need thereof an effective amount of the suspension. The composition of the present invention is useful in the management of one or more diseases/disorders which includes prophylaxis, amelioration and/or treatment of such disease(s) or disorder(s).

In a further embodiment is provided the use and method of using the suspensions of the present invention for the preparation of medicament for the treatment of one or more diseases or disorders selected from but not limited to a group comprising viral infections, bacterial infections, hypertension, treatment of heart failure, management of CNS disorders, epilepsy and various cardiovascular disorders which depends on the specific active agent used in the composition. In an embodiment, the suspensions of the present invention are intended for prophylactic or therapeutic use.

Stability studies were conducted on the paracetamol dual release suspension (Example 1) at room temperature for 2.5 months. Further the dissolution studies were carried out at different timescales on different days as shown in Table 1. The said dissolution profile data for the reconstituted suspension composition is further represented by FIG. 1.

TABLE 1

| | Initial | | 7th Day | | 14th Day | | 21st day | | 28th Day | | 1.5 Month | | 2.5 Month | |
|---|---|---|---|---|---|---|---|---|---|---|---|---|---|---|
| Time | Avg. | RSD | Avg. | RSD | Avg. | RSD | Avg. | RSD | Avg. | RSD | Avg. | RSD | Avg. | RSD |
| 1 hr | 47.3 | 5.4 | 43.9 | 2.9 | 43.8 | 14.3 | 42.4 | 5.5 | 46.4 | 5.7 | 40.4 | 11.8 | 48.2 | 0.3 |
| 2 hrs | 59.4 | 2 | 54.7 | 0.6 | 55.6 | 10.6 | 55.4 | 3 | 58.4 | 4.6 | 52.8 | 7.8 | 57.7 | 0.3 |
| 3 hrs | 67.6 | 1.2 | 62.5 | 0.4 | 60.5 | 8.1 | 62.4 | 1.2 | 64.5 | 3.6 | 61.2 | 5.6 | 64.8 | 1.4 |
| 4 hrs | 73.4 | 0.6 | 68.2 | 0.4 | 66.9 | 6.8 | 66.8 | 1 | 69.8 | 4 | 66.6 | 4.2 | 70.5 | 0.6 |
| 5 hrs | 78.6 | 3.1 | 73.3 | 1.5 | 72.6 | 4.7 | 71.8 | 2.5 | 75.5 | 0.9 | 70.6 | 3.7 | 76.2 | 1 |
| 6 hrs | 82 | 1.6 | 77.2 | 0.8 | 77 | 4.6 | 75.7 | 2.9 | 78.4 | 2.3 | 74.6 | 1.7 | 79.4 | 0.8 |
| 8 hrs | 88.1 | 1.3 | 82.8 | 0.9 | 83.4 | 4 | 81.8 | 0.8 | 85 | 2.4 | 81.5 | 2.1 | 85.6 | 0.8 |
| 10 hrs | 92.5 | 1.9 | 86.8 | 1 | 89.1 | 3.1 | 86.3 | 2 | 89.5 | 2.1 | — | — | — | — |
| 12 hrs | 97.2 | 1.7 | 91.6 | 1.7 | 93.4 | 1.7 | 90.4 | 2.3 | 93.1 | 3.5 | 88.2 | 0.9 | 95.3 | 2.1 |
| 16 hrs | 102.6 | 1.9 | 97 | 1.4 | 97.7 | 2 | 96.9 | 0.9 | 97.6 | 2 | 94.5 | 0.4 | 99.3 | 1.4 |
| 24 hrs | — | — | — | — | — | — | — | — | — | — | 99.2 | 0.6 | 103.8 | 1.7 |

The following chart gives the idea about the ability of the prevent invention in masking the taste of the bitter drugs.

### Taste Evaluation Study

### Taste Evaluation:

The formulation was prepared and its taste was evaluated by administering it to human volunteers. Seven subjects were chosen for the study. The volunteers were informed to rank the perception of the taste on the following scale Table 2.

TABLE 2
Rating Chart:

| Score | Inference |
|---|---|
| 1 | Highly Bitter Taste |
| 2 | Moderately Bitter Taste |
| 3 | Slightly Bitter Taste |
| 4 | No Bitter Taste |
| 5 | Acceptable Taste |
| 6 | Good Taste |

Each volunteer was made to rank the formulations on the above mentioned scale after taking 1 ml of the formulation orally. After taste evaluation, the data was evaluated using central tendency measure i.e. mean.

### Conclusions:

On observing the mean value for the formulation, it is found that Paracetamol CR Suspension with mean 5.857 was good in taste.

The examples given below serve to illustrate embodiments of the present invention. However they do not intend to limit the scope of the present invention.

### EXAMPLES

### Example-1

| | | |
|---|---|---|
| S. No. | Ingredient | Quantity/unit |

| A) | Core composition | | |
|---|---|---|---|
| 1. | Paracetamol | 250 | mg |
| 2. | Compritol ATO 888 | 250 | mg |
| B) | Coating dispersion-1 | | |
| 3. | Eudragit ® RS30D | 229.33 | mg |
| 4. | Dibutyl sebacate | 14.90 | mg |
| 5. | Talc | 29.75 | mg |
| 6. | Tween 80 | 0.16 | mg |
| 7. | Sunset yellow | 0.16 | mg |
| 8. | Purified water | q.s. (lost in processing) | |
| C) | Reconstitutable Suspension | | |
| | composition | | |
| 9. | Paracetamol | 100 | mg |
| 10. | Coated granules | 150 mg(equivalent base) | |
| 11. | Xanthan gum | 15 | mg |
| 12. | Sodium alginate | 50 | mg |
| 13. | Veegum | 10 | mg |
| 14. | Sorbitol | 125 | mg |
| 15. | Strawberry flavor | 0.75 | mg |
| 16. | Methyl paraben | 10 | mg |
| 17. | Colloidal silicon dioxide | 5 | mg |

| | | |
|---|---|---|
| 18. | Purified water | q.s. to 5 ml |

### Procedure:

i) Compritol ATO 888 was weighed and melted.
ii) Paracetamol was weighed and dispersed in the molten Compritol ATO 888 of step (i).
iii) The molten dispersion prepared in step (ii) was spreaded to form layers and allowed to congeal at room temperature.
iv) The congealed mass of step (iii) was passed through sieve # 10 (or equivalent sieve) followed by mesh # 40 (or equivalent sieve) in order to obtain granules.
v) The granules prepared in step (iv) were sifted through sieve #60 and were collected.
vi) The granules from step (v) were compacted using roll compactor and were kept at 50° C. for 24 hrs.
vii) Eudragit® RS30D, dibutyl sebacate, talc, Tween 80 and sunset yellow were weighed and dispersed in purified water.

viii) The coating dispersion of step (v) was filtered and the filtered material was coated onto the granules of step (vi).
ix) Paracetamol, xanthan gum, sodium alginate, veegum, sorbitol, methyl paraben and strawberry flavor were weighed together and mixed well with the granules obtained in step (viii).
x) The mixture obtained in step (ix) was blended with colloidal silicon dioxide and a suspension was obtained by adding and mixing with purified water q.s. to 5 ml.

Dissolution profile for the paracetamol reconstituted suspension for 2.5 months remains constant represented in FIG. 1.

### Example-2

| | | |
|---|---|---|
| S. No. | Ingredient | Quantity/unit |

| A) | Core composition | | |
|---|---|---|---|
| 1. | Paracetamol | 250 | mg |
| 2. | Eudragit RSPO | 37.5 | mg |
| 3. | Aerosil | 2.5 | mg |
| 4. | Isopropyl alcohol | | |
| B) | Coating dispersion-1 | | |
| 5. | Eudragit ® RS30D | 114.72 | mg |
| 6. | Dibutyl sebacate | 7.46 | mg |
| 7. | Talc | 14.88 | mg |
| 8. | Tween 80 | 0.08 | mg |
| 9. | Sunset yellow | 1.17 | mg |
| 10. | Purified water | q.s. (lost in processing) | |
| C) | Reconstitutable Suspension | | |
| | composition | | |
| 11. | Paracetamol | 80 | mg |
| 12. | Coated Granules | 232.5 | mg |
| 13. | Avicel CL611 | 8 | mg |
| 14. | Sodium Alginate | 50 | mg |
| 15. | Veegum | 50 | mg |
| 16. | Sorbitol | 250 | mg |
| 17. | Strawberry flavor | 0.75 | mg |
| 18. | Methyl paraben | 10.0 | mg |
| 19. | Colloidal silicon dioxide | 5 | mg |

| | | |
|---|---|---|
| 20. | Purified water | q.s. to 5 ml |

### Procedure:

i) Eudragit RSPO, paracetamol and aerosil were weighed and blended together.
ii) Step (i) material was granulated with isopropyl alcohol.
iii) Granulated mass was dried.
iv) The dried mass of step (iii) was passed through sieve # 10 (or equivalent sieve) followed by mesh # 40 (or equivalent sieve) in order to obtain granules.
v) Eudragit® RS30D, dibutyl sebacate, talc, Tween 80 and sunset yellow were weighed and dispersed in purified water.
vi) The coating dispersion of step (v) was filtered and the filtered material was coated onto the granules of step (vi).
vii) Paracetamol, Avicel CL611, sodium alginate, veegum, sorbitol, methyl paraben and strawberry flavor were weighed together and mixed well with the granules obtained in step (viii).
viii) The mixture obtained in step (ix) was blended with colloidal silicon dioxide and a suspension was obtained by adding and mixing with purified water q.s. to 5 ml.

### Example-3

| | | |
|---|---|---|
| S. No. | Ingredient | Quantity/unit |

| A) | Core composition | | |
|---|---|---|---|
| 1. | Naproxen | 125 | mg |
| 2. | Compritol ATO 888 | 125 | mg |
| B) | Coating dispersion-1 | | |
| 3. | Eudragit ®RL30D | 100.88 | mg |
| 4. | Dibutyl sebacate | 6.55 | mg |
| 5. | Talc | 3.06 | mg |
| 6. | Tween 80 | 0.07 | mg |
| 7. | Sunset yellow | 0.07 | mg |
| 8. | Purified water | q.s. (lost in processing) | |
| C) | Reconstitutable Suspension | | |
| | composition | | |
| 9. | Naproxen | 25 | mg |
| 10. | Coated Granules | 220 | mg |
| 11. | Xanthan Gum | 15 | mg |
| 12. | Sodium Alginate | 50 | mg |
| 13. | Veegum | 10 | mg |
| 14. | Strawberry flavor | 0.75 | mg |
| 15. | Methyl paraben | 10.0 | mg |
| 16. | Colloidal silicon dioxide | 5 | mg |

| | | |
|---|---|---|
| 17. | Purified water | q.s. to 5 ml |

### Procedure:

i) Compritol ATO 888 was weighed and melted.
ii) Naproxen was weighed and dispersed in the molten Compritol ATO 888 of step (i).
iii) The molten dispersion prepared in step (ii) was spreaded to form layers and allowed to congeal at room temperature.
iv) The congealed mass of step (iii) was passed through sieve # 10 (or equivalent sieve) followed by mesh # 40 (or equivalent sieve) in order to obtain granules.
v) The granules prepared in step (iv) were sifted through sieve #60 and were collected.
vi) The granules from step (v) were compacted using roll compactor and were kept at 50° C. for 24 hrs.

vii) Eudragit® RL30D, dibutyl sebacate, talc, Tween 80 and sunset yellow were weighed and dispersed in purified water.

viii) The coating dispersion of step (v) was filtered and the filtered material was coated onto the granules of step (vi).

ix) Naproxen, xanthan gum, sodium alginate, veegum, methyl paraben and strawberry flavor were weighed together and mixed well with the granules obtained in step (viii).

x) The mixture obtained in step (ix) was blended with colloidal silicon dioxide and a suspension was obtained by adding and mixing with Purified water q.s. to 5 ml.

### Example-4

| | | |
|---|---|---|
| S. No. | Ingredient | Quantity/unit |

| A) | Core composition | | |
|---|---|---|---|
| 1. | Amoxicillin | 400 | mg |
| 2. | Sterotex | 400 | mg |
| B) | Coating dispersion-1 | | |
| 3. | Eudragit ® RS30D | 402.28 | mg |
| 4. | Dibutyl sebacate | 26.32 | mg |
| 5. | Talc | 52.46 | mg |
| 6. | Tween 80 | 0.27 | mg |
| 7. | Sunset yellow | 0.27 | mg |
| 8. | Purified water | q.s. (lost in processing) | |
| C) | Reconstitutable Suspension | | |
| | composition | | |
| 9. | Coated Granules | 720 | mg |
| 10. | Xanthan gum | 15 | mg |
| 11. | Sodium alginate | 50 | mg |
| 12. | Veegum | 10 | mg |
| 13. | Sucrose | 500 | mg |
| 14. | Amoxicillin | 100 | mg |
| 15. | Strawberry flavor | 0.75 | mg |
| 16. | Methyl paraben | 10 | mg |
| 17. | Colloidal silicon dioxide | 10 | mg |

| | | |
|---|---|---|
| 18. | Purified water | q.s. to 5 ml |

### Procedure:

i) Sterotex was weighed and melted.
ii) Amoxicillin was weighed and dispersed in the molten Sterotex of step (i).
iii) The molten dispersion prepared in step (ii) was spreaded to form layers and allowed to congeal at room temperature.
iv) The congealed mass of step (iii) was passed through sieve # 10 (or equivalent sieve) followed by mesh # 40 (or equivalent sieve) in order to obtain granules.
v) The granules prepared in step (iv) were sifted through sieve #60 and were collected.
vi) The granules from step (v) were compacted using roll compactor and were kept at 50° C. for 24 hrs.
vii) Eudragit® RS30D, dibutyl sebacate, talc, Tween 80 and sunset yellow were weighed and dispersed in purified water.
viii) The coating dispersion of step (v) was filtered and the filtered material was coated onto the granules of step (vi).
ix) Amoxicillin, xantural 180, sodium alginate, sucrose, veegum, methyl paraben and strawberry flavor were weighed together and mixed well with the granules obtained in step (viii).
x) The mixture obtained in step (ix) was blended with colloidal silicon dioxide and a suspension was obtained by adding and mixing with purified water q.s. to 5 ml.

### Example-5

| | | |
|---|---|---|
| S. No. | Ingredient | Quantity/unit |

| A) | Core composition | | |
|---|---|---|---|
| 1. | Ciprofloxacin | 500 | mg |
| 2. | Compritol ATO 888 | 500 | mg |
| B) | Coating dispersion-1 | | |
| 3. | Eudragit ® RS30D | 443.52 | mg |
| 4. | Dibutyl sebacate | 28.87 | mg |
| 5. | Talc | 57.49 | mg |
| 6. | Tween 80 | 0.29 | mg |
| 7. | Sunset yellow | 0.29 | mg |
| 8. | Purified water | q.s. (lost in processing) | |
| C) | Reconstitutable Suspension | | |
| | composition | | |
| 9. | Coated Granules | 920 | mg |
| 10. | Xanthan gum | 15 | mg |
| 11. | Sodium Alginate | 50 | mg |
| 12. | Veegum | 10 | mg |
| 13. | Sucrose | 500 | mg |
| 14. | Ciprofloxacin HCl | 110.4 | mg |
| 15. | Strawberry flavor | 0.75 | mg |
| 16. | Methyl paraben | 10 | mg |
| 17. | Colloidal silicon dioxide | 10 | mg |

| | | |
|---|---|---|
| 18. | Purified water | q.s. to 5 ml |

### Procedure:

i) Compritol ATO 888 was weighed and melted.
ii) Ciprofloxacin was weighed and dispersed in the molten Compritol ATO 888 of step (i).
iii) The molten dispersion prepared in step (ii) was spreaded to form layers and allowed to congeal at room temperature.
iv) The congealed mass of step (iii) was passed through sieve # 10 (or equivalent sieve) followed by mesh # 40 (or equivalent sieve) in order to obtain granules.
v) The granules prepared in step (iv) were sifted through sieve #60 and were collected.
vi) The granules from step (v) were compacted using roll compactor and were kept at 50° C. for 24 hrs.
vii) Eudragit® RS30D, dibutyl sebacate, talc, Tween 80 and sunset yellow were weighed and dispersed in purified water.
viii) The coating dispersion of step (v) was filtered and the filtered material was coated onto the granules of step (vi).
ix) Ciprofloxacin HCl, xanthan gum, sodium alginate, sucrose, veegum, methyl paraben and strawberry flavor were weighed together and mixed well with the granules obtained in step (viii).
x) The mixture obtained in step (ix) was blended with colloidal silica and a suspension was obtained by adding and mixing with purified water q.s. to 5 ml.

### Example-6

### Powder for Reconstitution

| | | |
|---|---|---|
| S. No. | Ingredient | Quantity/unit |

| A) | Core composition | | |
|---|---|---|---|
| 1. | Paracetamol | 250 | mg |
| 2. | Compritol ATO 888 | 250 | mg |
| B) | Coating dispersion-1 | | |
| 3. | Eudragit ® RS30D | 272.14 | mg |
| 4. | Dibutyl sebacate | 17.69 | mg |
| 5. | Talc | 35.30 | mg |
| 6. | Tween 80 | 0.19 | mg |
| 7. | Sunset yellow | 0.19 | mg |
| 8. | Purified water | q.s. (lost in processing) | |
| C) | Reconstitutable Suspension | | |
| | composition | | |
| 9. | Paracetamol | 100 | mg |
| 10. | Coated Granules | 150 mg(equivalent base) | |
| 11. | Xanthan gum | 15 | mg |
| 12. | Sorbitol | 125 | mg |
| 13. | Sucrose | 750 | mg |
| 14. | Strawberry flavor | 0.75 | mg |
| 15. | Sodium benzoate | 10.0 | mg |
| 16. | Colloidal silicon dioxide | 5 | mg |

### Procedure:

xi) Compritol ATO 888 was weighed and melted.
xii) Paracetamol was weighed and dispersed in the molten Compritol ATO 888 of step (i).
xiii) The molten dispersion prepared in step (ii) was spreaded to form layers and allowed to congeal at room temperature.
xiv) The congealed mass of step (iii) was passed through sieve # 10 (or equivalent sieve) followed by mesh # 40 (or equivalent sieve) in order to obtain granules.
xv) The granules prepared in step (iv) were sifted through sieve #60 and were collected.
xvi) The granules from step (v) were compacted using roll compactor and were kept at 50° C. for 24 hrs.
xvii) Eudragit® RS30D, dibutyl sebacate, talc, Tween 80 and sunset yellow were weighed and dispersed in purified water.
xviii) The coating dispersion of step (v) was filtered and the filtered material was coated onto the granules of step (vi).
xix) Paracetamol, xantural 75, sucrose, sorbitol, sodium benzoate and strawberry flavor were weighed together and mixed well with the granules obtained in step (viii).
xx) The mixture obtained in step (ix) was blended with colloidal silicon dioxide.

1. Oral dual-release pharmaceutical suspension comprising
a. an immediate release fraction and a sustained release fraction of the active agent in the range from about 1% to about 70% by weight of final composition or its pharmaceutically acceptable salts, derivatives, isomers, polymorphs, solvates, hydrates, analogues, enantiomers, tautomeric forms or mixtures thereof;
b. wherein immediate release fraction is in form of solution, suspension or uncoated microgranules;
c. sustained release fraction is in the form of suspended coated microparticles which are suspended in the aqueous suspending medium comprising a core and at least one coat, wherein the core comprises at least one active agent(s), optionally at least one water-insoluble polymer(s) and optionally one or more pharmaceutically acceptable excipient(s); and at least one coat comprising at least one pH independent water-insoluble polymer(s) along with one or more pharmaceutically acceptable excipient(s), and wherein the coating composition may constitute from about 5% to about 50% by weight of the total sustained release microparticle weight and wherein immediate release fraction to extended release fraction is in a fixed ratio of about 20:1 to about 1:20, in admixture with one or more pharmaceutically acceptable excipient(s).

2. Oral dual-release pharmaceutical suspension according to claim 1 the size of the core microparticles may range from about 0.5 μm to about 2000 μm or from about 1 μm to about 1000 μm or from about 50 μm to about 500 μm.

3. A composition according to claim 1, wherein the composition is a dual release suspension comprising of at least one active agent(s) selected preferably from a group comprising paracetamol, nimesulide, diclofenac sodium, indomethacin, ketoprofen, diflunisal, piroxicam, naproxen, levocetirizine, desloratadine, fexofenadine, aspirin, glipizide, glyburide, glimepiride, gliclazide, metformin, rosiglitazone, pioglitazone, vildagliptin, sitagliptin, metoclopramide, diphenhydramine, loratadine, desloratadine, meclizine, quetiapine, fexofenadine, pheniramine, cetirizine, promethazine, chlorpheniramine, cimetidine, famotidine, ranitidine, nizatidine, roxatidine, lafutidine, metronidazole, chlorpromazine, fluphenazine, prochlorperazine, rimantadine, amantadine, emtricitabine, lamivudine, zidovudine, stavudine, zalcitabine, ritonavir, saquinavir, indinavir, nevirapine, ciproflocaxin, amoxicillin, voriconazole, posaconazole, oxcarbazepine, carbamazepine, phenyloin, indinavir, nevirapine, ciproflocaxin, amoxicillin and the like or pharmaceutically acceptable salts, hydrates, polymorphs, esters and derivatives thereof used either alone or in combination thereof.

4. The composition according to claim 1, wherein the pH independent polymer water-insoluble in the range of from about 5% to about 30% by weight of the total composition selected from a group comprising polyacrylate polymers, Eudragit® RS, Eudragit® RSPO, Eudragit® RL or a cellulosic polymer e.g. ethyl cellulose, hydroxyl ethyl cellulose, cellulose acetate or mixtures thereof.

5. The composition according to claim 1, wherein solubility modifying agents in the range from about 5% to about 50% by weight of the final composition are selected from a group comprising sugars, partially hydrolyzed starch solids, partially hydrolyzed corn syrup solids, sorbitol, xylitol, mannitol, amino acids, weak acids and the like.

6. The composition according to claim 1, wherein the lipidic agent present in the core composition of the microparticle in the range from about 5% to about 30% by weight of the final composition is selected from but not limited to a group comprising beeswax, carnauba wax, cetyl palmitate, Compritol® 888 ATO (glyceryl behenate), glyceryl monostearate, precirol ATO (glyceryl palmitostearate), hydrogenated castor oil, paraffin wax, stearic acid, steryl alcohol, glyceryl trimyristate (Dynasan 114), gelucire, Sterotex® K or mixtures thereof.

7. The composition according to claim 1, wherein the pharmaceutically acceptable excipient(s) are selected from to a group comprising viscosity modifiers, anti-caking agents, in-situ gelling agent, thixotropic agents, anti-oxidants, colorants, flavoring agents, sweeteners, preservatives, glidants, chelating agents, plasticizers, vehicles, wetting agents, complexing agents, buffering agents, preservatives, suspending agents, release modifiers used either alone or in combination thereof.

8. The composition according to claim 1, wherein the composition is the taste masking dual release pharmaceutical formulations for oral administration of active agents which produces unpleasant or bitter taste in mouth of a subject upon administration.

9. Oral dual-release pharmaceutical suspension according to claim 1, wherein the sustained release microgranules of the drug are suspended in the aqueous suspending medium comprising drug or uncoated microgranules of the drug in such a way that the diffusion of the drug from sustained release microgranules to the aqueous suspending medium is negligible leading to a stable dissolution profile over an extended period of time.

10. A process for preparation of compositions according to claim 1, wherein powder or granular formulations may be manufactured using hot melt granulation techniques, in which drug is dispersed/dissolved in hot molten lipid and after drying, granules are sized to the required range.

11. A process for preparation of compositions according to claim 1, which comprises of the following steps:
i) preparation of microparticles comprising a core and at least one coat,
ii) Suspending the composition of step (i) in a suitable medium containing immediate release fraction in solution form, suspended form or in form of uncoated microparticles to obtain a dual-release suspension.

12. A composition according to claim 1, wherein the composition is in preferably in the form of dual-release suspension which is either in the form of ready to use suspension or in the form of powder ready for reconstituted prior to use.

13. A method of using the pharmaceutical composition according to claim 1, for the for the treatment of one or more diseases or disorders selected from but not limited to a group comprising viral infections, bacterial infections, hypertension, treatment of heart failure, management of CNS disorders, epilepsy and various cardiovascular disorders which depends on the specific active agent used in the composition.

14. Use of the pharmaceutical composition according to claim 1, for the for the treatment of one or more diseases or disorders selected from but not limited to a group comprising viral infections, bacterial infections, hypertension, treatment of heart failure, management of CNS disorders, epilepsy and various cardiovascular disorders which depends on the specific active agent used in the composition.

15. The pharmaceutical composition according to claim 1, for the preparation of medicament for the treatment of one or more diseases or disorders selected from but not limited to a group comprising viral infections, bacterial infections, hypertension, treatment of heart failure, management of CNS disorders, epilepsy and various cardiovascular disorders which depends on the specific active agent used in the composition.

16. The pharmaceutical compositions substantially as herein described and illustrated by the examples.

17. The processes for the preparation of pharmaceutical compositions substantially as herein described and illustrated by the examples.
